(19) United States
(12) Patent Application Publication
(10) Pub. No.: US 2008/0293955 A1
(43) Pub. Date: Nov. 27, 2008

(54) Method for Producing Vitamin E-Adsorbates

(75) Inventors: Robert Rühle, Carlsberg (DE); Dieter Feuerstein, Dudenhofen (DE); Wolfram Strauch, Hamburg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/158,495

(22) PCT Filed: Dec. 11, 2006
(86) PCT No.: PCT/EP06/69524

§ 371 Date: Jun. 20, 2008

(30) Foreign Application Priority Data
Dec. 22, 2005 (DE) 102005062009.4-41

(51) Int. Cl. C07D 311/72

(52) U.S. Cl. 549/408

(57) ABSTRACT

The present invention relates to a process for preparing adsorbates of at least one vitamin E compound in adsorbed form on a silica support, comprising, as the essential step, the mixing of the corresponding vitamin E compound with the silica support under reduced pressure. The invention further relates to adsorbates which are preparable by the process mentioned.

### TECHNICAL FIELD OF THE INVENTION

The present invention relates to a process for preparing adsorbates of at least one vitamin E compound in adsorbed form on a silica support, comprising, as the essential step, the mixing of the corresponding vitamin E compound with the silica support under reduced pressure. The invention further relates to adsorbates which are preparable by the process mentioned.

The nutritional quality of compound feed is determined crucially by its content of essential ingredients, for example mineral substances, trace elements, amino acids or vitamins. The particular amounts of the ingredients mentioned in feeds can be adjusted to the particular requirements within wide limits by adding supplements or additives. The supplements or additives used comprise the desired ingredients or else a plurality of different ingredients in a form which is suitable for producing feeds. One example of these is vitamin E acetate supported on silica get, which is obtainable, for example, under the name Lutavit® E 50 (BASF Aktiengesellschaft).

Owing to the increasing significance of compound feeds in animal nutrition, there is a continuing need for inexpensive, industrially easily obtainable supplements or additives which meet the requirements for processing in feeds. In addition, there is a constant need for improved processes for economically viable preparation of the additives for feeds mentioned.

### STATE OF THE ART

EP 1 018 303 discloses active ingredient adsorbates based on silicas, which comprise precipitated silica and an active ingredient, for example vitamin E acetate, in an amount of from 1 to greater than 60% by weight. Also disclosed is a process for preparing the adsorbates mentioned, which envisages the granulation of an aqueous suspension of precipitated silica and the appropriate active ingredient in a fluidized bed. However, this process is associated with a high level of energy demands and hence cost.

EP 0 984 772 relates to compositions comprising at least one liquid, for example choline hydrochloride or vitamin E, which is absorbed on a support comprising precipitated silica, the precipitated silica being present in roughly spherical form, the spheres having a mean size of over 150 μm, a bulk density of less than 0.29; a residue rate in a sieve of mesh width 75 μm of at least 88% and a specific pore volume. The compositions mentioned can be prepared by mixing the appropriate liquid with the support in a conventional manner.

EP 0 966 207 relates to compositions comprising at least one liquid, for example choline hydrochloride or vitamin E, which is absorbed on a support comprising precipitated silica, the precipitated silica being present in roughly spherical form, the spheres having a mean size of over 150 μm, a bulk density of less than 0.29; a residue rate in a sieve of mesh width 75 μm of at least 92% and a DOP oil number of at least 250 ml/100 g. The compositions mentioned can be prepared by mixing the appropriate liquid with the support in a conventional manner. They may have a liquid content of from 50 to 65% by weight.

EP 0 345 109 discloses specific precipitated silicas which feature a certain BET surface area, a certain DOP oil absorption, a certain minimum bulk density, a mean particle diameter between 80 and 150 μm and a certain granulometric dispersion index. Additionally disclosed are prepared compositions which, in addition to the support, comprise an absorbed liquid, for example vitamin E, and a process for preparing the specific precipitated silicas mentioned. The absorption should be undertaken in a conventional manner in a mixer.

In Mühle+Mischfutter 138 (8), 2001, p. 262-264, W. Strauch describes the preparation of energy-rich pelletized poultry feed by vacuum coating. By using a vacuum mixer and spraying on mixed fat, a pelletized, wheat-based broiler feed with a total fat content of 10% is prepared.

EP 0 556 883 discloses a process for preparing extruded feed pellets laden with an active ingredient. After the extrusion, the feed pellets are admixed in a fluid phase with the active ingredient, which would have been damaged by the extrusion process, and loaded under reduced pressure. The active ingredient is an enzyme; protein, pigment, vitamin, antioxidant, dye or a carotenoid.

WO 00/27362 relates to a process for preparing dry, free-flowing vitamin powders by mixing dried corn starch, silica and at least one vitamin in a mixer. First, corn starch and silica are mixed, and then the vitamin is added. The mixing is preferably carried out in a mixer with a speed of at least 3600 rpm. Preferred vitamins mentioned include vitamin E, which is preferably used in an amount of from 50 to 80% by weight.

One process for preparing dry, fine and free-flowing powder comprising vitamin E or vitamin E acetate is disclosed by U.S. Pat. No. 4,603,143. The process comprises the addition of a silicon-containing adsorbent in the form of substantially discrete, nonamorphous agglomerates having a minimum length and/or width of 300 μm in a vessel and subsequently adding vitamin E or vitamin E acetate in liquid form in an amount which is sufficient to obtain an about 40 to about 60% strength by weight powder with simultaneous mixing.

### OBJECT OF THE INVENTION

It was an object of the present invention to provide an economically viable process which allows the provision of adsorbates of vitamin E compounds on suitable supports, which feature a maximum content of adsorbed vitamin E compound and which additionally have at least comparably good material properties, for example good free-flow and a low tendency to adhesion, such as the corresponding adsorbates prepared by known processes.

### DESCRIPTION OF THE INVENTION AND THE PREFERRED EMBODIMENTS

The object is achieved in accordance with the invention by the provision of a process for preparing adsorbates of at least one vitamin E compound of the formula (I)

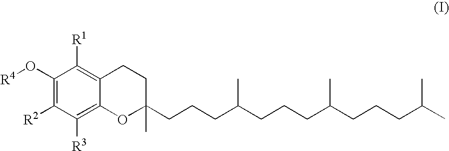

where
R¹, R², R³ are each independently hydrogen or methyl and
R⁴ is hydrogen or C₂- to C₁₂-acyl
in adsorbed form on a silica support, comprising the mixing of the at least one vitamin E compound of the formula (I) with the silica support under reduced pressure.

The process according to the invention is suitable in particular for preparing those adsorbates which consist to an extent of at least 90% by weight, preferably to an extent of at least 95% by weight and more preferably to an extent of at least 98% by weight of at least one, i.e. of preferably one, vitamin E compound or a mixture of a plurality of, generally from 2 to about 4, in particular 2 or 3, different vitamin E compounds of the formula (I) and a silica support. In addition to the ingredients mentioned, the inventive adsorbates may also comprise small amounts of water or other impurities, for example by-products or impurities from the preparation or the isolation or purification of the vitamin E compound or compounds used in accordance with the invention.

The adsorbates preparable by the process according to the invention are preferably notable in that, based on the amount of the finished adsorbate, they consist to an extent of from about 30 to about 70% by weight, preferably to an extent of from at least about 40 to about 65% by weight and more preferably to an extent of from about 50 to about 65% by weight of a vitamin E compound of the formula (I).

In the context of the present invention, the term adsorbate is to be interpreted broadly and refers generally to mixtures of the particular vitamin E compound or vitamin E compounds with the silica support used, the particular vitamin E compound having been applied to the surface of the silica support in the administration form used in each case. In the case of porous forms of the silica support, the particular vitamin E compound can accordingly also be introduced into the cavities present in the silica support.

Suitable vitamin E compounds to be adsorbed in the process according to the invention are those of the general formula (I)

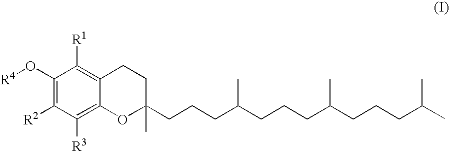

in which the R¹, R² and R³ radicals may each independently be hydrogen or methyl, and the R⁴ radical is hydrogen or a C₂- to C₁₂-acyl group. Vitamin E compounds of the formula (I) preferred in accordance with the invention are those in which the R¹, R² and R³ radicals are each a methyl group.

The R⁴ radical is preferably a straight-chain or branched acyl radical having from 2 to 6 carbon atoms, especially preferably acetyl. Accordingly, the adsorbates to be prepared with preference in the process according to the invention are those in which the vitamin E compound is present in the form of an acetate. A vitamin E compound particularly preferred in the process according to the invention is α-tocopherol acetate.

The vitamin E compounds present in the adsorbates to be prepared in accordance with the invention may each be isolated from natural sources or prepared by suitable synthesis processes. Preference is given to those vitamin E compounds which are prepared synthetically. Enantiomerically pure vitamin E compounds and racemic mixtures or mixtures of different diastereomers with regard to the stereogenic centers of the particular vitamin E compound or compounds are equally suitable as feedstocks in the process according to the invention, Preference is given in accordance with the invention to those vitamin E compounds which are present in liquid or oily form at room temperature.

In a preferred embodiment, a suitable vitamin E compound as a starting material in the process according to the invention is synthetic dl-α-tocopherol acetate, which is typically obtained in the form of an oil with a content of from about 90 to about 95% by weight of dl-α-tocopherol acetate (referred to hereinafter as “Vitamin E oil”). Like the other vitamin E compounds usable in accordance with the invention, it may also comprise solvent residues to a small degree, for example from the preparation or the isolation of the vitamin E compounds used in each case.

The second starting material in the process according to the invention is a silica support to which the selected vitamin E compound or compounds is/are absorbed in the inventive manner. The term silica support is to be interpreted broadly and refers to a fine support material based on silica, which is suitable for use as an adsorbent. Examples of silica supports include silica gels, precipitated silicas or else silicas obtained by flame hydrolysis. Additionally suitable as silica supports are also minerals which consist essentially of silicon dioxide, for example silica earths or kieselguhr, if suitable for use as a feed additive. However, the term silica support in the context of the present invention does not comprise the silicate vermiculite. Silica supports preferred in accordance with the invention are the precipitated silicas.

Silica supports preferred as a starting material in the process according to the invention have a mean particle size of from about 50 to about 600 μm, preferably from about 150 to about 400 μm, and a specific surface area of from about 50 to about 500 m²/g, preferably from about 100 to about 300 m²/g.

Precipitated silicas to be used with particular preference in the process according to the invention are, for example, those commercially available under the name Sipernat® 2200 (Degussa AG). In addition, examples of silica supports which are likewise preferred include: Sipernat® 2300 (Degussa AG), Tixosil® (Rhodia).

The process according to the invention comprises, as the essential step, the mixing of the at least one vitamin E compound of the formula (I) with the silica support under reduced pressure.

For the performance of the process according to the invention, suitable apparatus includes commercial vacuum mixers, for example the batchwise twin-shaft paddle mixer VC-450 from Dinnissen (Sevenum, the Netherlands) or rotating vacuum mixers from Forberg International AS, for example the model of the F-6-RVC design, which are available in various sizes up to a capacity of from 2000 to 7000 liters (F-5000-RVC) or comparable apparatus. The procedure for vacuum mixing corresponds substantially to the procedure known to those skilled in the art for vacuum coating of pelletized feeds, as described, for example, in the literature reference mentioned at the outset Mühle+Mischfutter 138 (8), 2001, p. 262-264, or else in Matrix Forum, March 2002 under the title “Processing. High energy poultry feed” by W. Strauch.

A vacuum mixer to be used advantageously has a horizontal casing and bottom flaps which appropriately open over the entire mixer length and one or more horizontal mixing rotors. The rotors preferably have oblique paddles, as a result of which the transport and exchange of the mixture particles during the mixing operation is ensured.

In the case of a usable volume of the vacuum mixer of, for example, about 450 l, the maximum usable mass per mixing operation is typically about 250 kg. The drive power of the mixer is then preferably, depending on the design of the mixer, up to about 5.5 kW.

The vacuum mixer selected advantageously has one or more, in general, depending on the size of the mixer selected, from about 1 to 10, nozzles, for example one-substance flat-jet nozzles, full-cone nozzles or hollow-cone nozzles, preferably one-substance flat-jet nozzles, which are typically integrated in the lid of the mixer.

The preparation process according to the invention is advantageously carried out in such a way that the selected silica support, preferably the selected precipitated silica, is introduced into the mixer and it is sealed tight. Subsequently, appropriately with a suitable vacuum pump, a reduced pressure is generated in the vacuum mixer. Preference is given to generating a reduced pressure in the vacuum mixer of from about 0.1 to about 0.5 bar (absolute), preferably from about 0.15 to about 0.3 bar (absolute) and more preferably from about 0.15 to about 0.25 bar (absolute). The power of the vacuum pump used is advantageously selected such that, from an economic point of view, advantageous evacuation times of up to about 15 min, preferably up to about 5 min and more preferably up to about 1 min are achieved. In batchwise operation, this ensures satisfactory conversion.

After the desired pressure has been established in the vacuum mixer or, if desired, even as early as during the pressure adjustment, the stirrer can be put into operation. Advantageous speeds of the stirrer have been found to be in the range from about 10 to 100 revolutions per minute (rpm), preferably from about 10 to about 50 rpm and more preferably from about 15 to about 40 rpm, depending on the selected design of the mixer.

Thereafter, the vitamin E compound to be adsorbed is added at reduced pressure to the silica support initially charged in the vacuum mixer. Preferably, the α-tocopherol acetate is appropriately sprayed through the aforementioned nozzles onto the stirred silica support. The spray pressure is advantageously from about 1 to about 15 bar, preferably from about 2 to about 10 bar. In addition, it has been found to be advantageous to add the vitamin E compound to be sprayed on or to spray it onto the support in the heated state, advantageously in the case of vitamin E oil, for example, preheated to from about 40 to about 100° C., preferably to from about 50 to about 90° C. and more preferably to from about 60 to about 90° C.

The spray process is typically complete after from about 1 to about 15 min, often after up to about 10 min. The vacuum mixer is then advantageously vented at reduced speed of the stirrer slowly until full pressure equalization, i.e. over a period of from about 0.5 to about 10 min, preferably from 1 to about 10 min and more preferably over a period of from about 1 to about 3 min. Finally, the adsorbate thus obtained can be removed from the mixer, for example by opening the bottom flaps, and be treated further as desired.

The preparation process according to the invention is notable in particular for simple industrial performability and short cycle times, as a result of which it can also be employed as a batchwise process in an economically advantageous manner.

The adsorbates prepared by the preparation process according to the invention are typically obtained in the form of stable, dry and fine powders with a mean particle diameter of from about 0.15 to about 0.6 mm, preferably from about 0.3 to about 0.6 mm. Typically, depending on the type and properties of the silica support used, the pulverulent adsorbates obtained are preferably those with the following particle size distribution: 5% by weight of the particles have a mean diameter of up to 100 μm, about 40% by weight of the particles have a mean diameter of up to 500 μm, about 95% by weight of the particles have a mean diameter of up to 2000 μm, preferably up to 1500 μm and more preferably up to 1000 μm, and less than 1% by weight of the particles have a mean diameter of 2000 μm or higher.

The adsorbates preparable by the process according to the invention, especially those which have precipitated silica as a support, are notable for advantageous substance properties, for example a high mechanical stability, high storage stability and good flow properties, i.e. low tack even at a high content of adsorbed vitamin E compound, and also a high bulk density or tapped density.

The present invention accordingly also relates to adsorbates of at least one vitamin E compound of the formula (I)

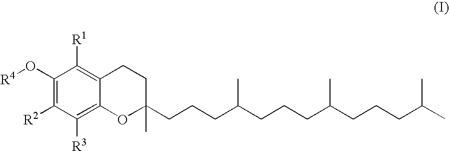

where
R¹, R², R³ are each independently hydrogen or methyl and
R⁴ is hydrogen or C₂- to C₁₂-acyl
in adsorbed form on a silica support, which are preparable by a process as described above which comprises the mixing of the at least one vitamin E compound of the formula (I) with the silica support under reduced pressure.

In a preferred embodiment, the present invention relates to adsorbates of α-tocopherol acetate in adsorbed form on a precipitated silica as a support, which are preparable by a process as described above which comprises the mixing of α-tocopherol acetate with the precipitated silica under reduced pressure.

Such adsorbates have a higher content of vitamin E compound used in each case in the pores of the silica support used in each case than adsorbates prepared by conventional processes. This means that, in the case of the inventive adsorbates, a larger ratio of the amount of vitamin E compound adsorbed into the pores to the amount of the silica support used in each case on the outer surface is present, which is manifested by a higher density of the inventive adsorbates with otherwise advantageous material properties such as good free-flow and low tack.

The inventive adsorbates can be transferred and stored in a customary manner and are notable for good storage stability. In some cases, it has been found to be advantageous to provide for a short maturing time of from about 10 min to about 30 min before the transfer, for example into sacks.

The silica supports mentioned which can be used in the process according to the invention, in particular the precipitated silicas, are additionally safe from a nutritional point of view and are therefore suitable for use as additives for foods or feeds.

### EXAMPLES

The following examples serve to illustrate the invention without restricting it in any way:

Bulk densities were determined to DIN 1060 with the influx instrument according to Böhme. Tapped densities were determined to DIN 53 194 with the tap volumeter according to Becker-Rosenmüller.

To determine angles of repose, the bulk material was piled up through a funnel with sieve to give a cone. The angle of repose reported was calculated by trigonometric means from the ratio of height to radius of the bulk material cone.

The flow behavior (efflux behavior) was determined with a cylindrical model vessel with variable aperture opening, which was filled from a fixed height with a constant mass of test material in each case. The smallest aperture for which the test material flowed out without bridge formation is measured.

### Example 1

A batchwise twin-shaft paddle mixer VC-450 from Dinnissen (Sevenum, the Netherlands) was charged with 100 kg of Sipernat® 2200 (Degussa AG) at a temperature of 19° C. Subsequently, the mixer was closed and evacuated to a pressure of 0.2 bar (abs.).

At this pressure, the mixer was started at a speed of 30 min⁻¹ and 166.7 kg of vitamin E oil preheated to 85.6° C. (92% by weight of racemic vitamin E acetate), corresponding to 62.5% by weight of the total amount of vitamin E oil and support, were sprayed on through four VeeJet® 9570 one-substance flat-jet nozzles (bore 5.2 mm, from Spraying Systems Co.) at a spray pressure of 8 bar within 5.5 min.

After the spray application process had ended, the speed of the mixer was halved and the mixer was vented. 166.7 kg of a vitamin E acetate adsorbate on Sipernat 22 were obtained with a bulk density of 0.607 g/cm³, a tapped density of 0.676 g/cm³ and an angle of repose of 25.0°. The resulting adsorbate is fine, dry and free-flowing. The flow behavior is characterized by an efflux time of 4.4 s for an aperture diameter of 30 mm.

### Example 2

The material obtained according to Example 1 was packaged in plastic bags and stored at room temperature for 3 weeks, and the flow behavior was investigated at weekly intervals. After one week of storage, the efflux time was 4.4 s, after two weeks 4.1 s and after four weeks 4.2 s, in each case at aperture diameter 30 mm.

### Comparative Example 3

A single-shaft shovel mixer (from Drais, Mannheim) with a usable volume of 25 l was initially charged with 6.0 kg of Sipernat® 2200 (Degussa AG) with a temperature of 18.0° C. At a speed of the mixer of 190 min⁻¹, 8.1 kg of vitamin E oil preheated to 65.2° C. (92% by weight of racemic vitamin E acetate), corresponding to 57.5% by weight of the total amount of vitamin E oil and support, were sprayed on at a spray pressure of 2.9 bar within 8 min. A tacky product with a tendency to form lumps was obtained.

1-16. (canceled)

17. A process for preparing adsorbates of at least one vitamin E compound of formula (I) 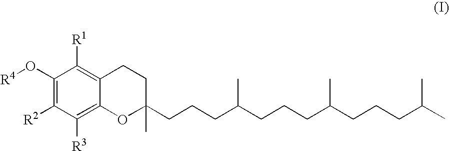
wherein
R¹, R², and R³ are each independently hydrogen or methyl; and
R⁴ is hydrogen or C₂- to C₁₂-acyl;
in adsorbed form on a silica support, comprising mixing at least one vitamin E compound of formula (I) with a silica support under reduced pressure.

18. The process of claim 17, wherein said silica support is a precipitated silica.

19. The process of claim 17, wherein said silica support is a silica gel.

20. The process of claim 17, wherein said adsorbates comprise at least 90% by weight of said at least one vitamin E compound of formula (I) and said silica support.

21. The process of claim 17, wherein said adsorbates comprise from 30% to 70% by weight of said at least one vitamin E compound of formula (I), based on the amount of the finished adsorbate.

22. The process of claim 17, wherein said adsorbates comprises from 50% to 65% by weight of said at least one vitamin E compound of formula (I), based on the amount of the finished adsorbate.

23. The process of claim 17, wherein said at least one vitamin E compound of formula (I) is α-tocopherol acetate.

24. The process of claim 17, wherein said adsorbate is obtained in the form of a fine and free-flowing powder.

25. The process of claim 24, wherein at least 95% by weight of the grains of said powder have a mean diameter of up to 2000 μm.

26. The process of claim 17, wherein the mixing of said at least one vitamin E compound of formula (I) with said silica support is carried out in a vacuum mixer.

27. The process of claim 17, wherein the mixing of said at least one vitamin E compound of formula (I) with the silica support is carried out at a pressure of from 0.15 to 0.25 bar.

28. The process of claim 17, wherein said at least one vitamin E compound of formula (I) is heated to a temperature of from 50° C. to 90° C. and mixed with said silica support.

29. The process of claim 26, wherein the mixing of said at least one vitamin E compound of the formula (I) with said silica support in said vacuum mixer is followed by venting said mixer over a period of from 1 to 3 minutes until full pressure equalization.

30. An adsorbate of at least one vitamin E compound of formula (I) 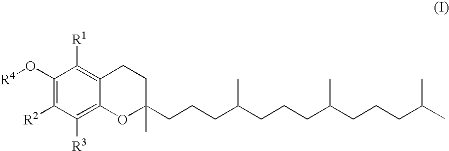
wherein
R¹, R², and R³ are each independently hydrogen or methyl; and
R⁴ is hydrogen or C₂- to C₁₂-acyl;
in adsorbed form on a silica support, prepared by the process of claim 17.

31. The adsorbate of claim 30, wherein said at least one vitamin E compound is α-tocopherol acetate.

32. The adsorbate of claim 30, wherein said silica support is a precipitated silica.
